(19) United States
(12) Patent Application Publication
(10) Pub. No.: US 2021/0008052 A1
(43) Pub. Date: Jan. 14, 2021

(54) ACTIVE INGREDIENT WOUNDCARE AND SCAR MANAGEMENT PRODUCTS, PROCESS OF MANUFACTURE AND USEFUL ARTICLES THEREOF

(71) Applicants: Mark E. Dillon, Center Valley, PA (US); Sean Michael Adams, Allentown, PA (US)

(72) Inventors: Mark E. Dillon, Center Valley, PA (US); Sean Michael Adams, Allentown, PA (US)

(73) Assignee: Bio Med Sciences, Inc., Allentown, PA (US)

(21) Appl. No.: 15/930,819

(22) Filed: May 13, 2020

Related U.S. Application Data
(60) Provisional application No. 62/847,115, filed on May 13, 2019.

(51) Int. Cl. A61K 31/4412; A61K 9/00; A61K 47/34; A61K 9/70; A61F 13/02; B32B 27/28; B32B 27/12; B32B 27/06; B32B 5/02; B32B 37/15

(52) U.S. Cl. CPC A61K 31/4412; A61K 9/0014; A61K 47/34; A61K 9/7007; A61F 13/0283; B32B 2535/00; B32B 27/12; B32B 27/065; B32B 5/022; B32B 5/024; B32B 37/15; B32B 27/283

(57) ABSTRACT

This invention relates to a novel method of reducing dermal scars resulting from various types of dermal injuries. Particularly, this invention relates to wound dressings and to scar management products each of which incorporate an active pharmaceutical ingredient to reduce and/or prevent dermal scarring.

### BACKGROUND OF THE INVENTION

### 1. Field of the Invention

This invention relates to novel wound dressings and scar management products. Particularly, this invention relates to wound dressings which incorporate an active pharmaceutical ingredient (“API”), and scar management products also containing an API. In each case, the API is delivered to the wound or scar area to reduce and prevent dermal scarring.

### 2. Description of the Prior Art

It is well-known that fibroblast cells are a major contributor to scar formation. Reducing the activity of such cells has the potential to significantly reduce dermal scarring due to injury or surgery.

Bio Med Sciences, Inc. of Allentown, Pa., manufactures and markets the Silon-TSR® and Rylon® brands of wound dressings. Additionally, Bio Med Sciences, Inc. manufactures and markets the Silon® and Oleeva® brands of scar management products including patches and splinting materials.

Pirfenidone is a drug that is currently approved by the U.S. Food and Drug Administration (“FDA”) to treat pulmonary fibrosis, a disease wherein fibroblasts accumulate in lung tissue and differentiate into myofibroblasts that secrete collagen and other proteins. This same biophysiological mechanism is known to induce scar formation in dermal wounds. Others have described in the literature the use of 8% Pirfenidone gel applied topically to ameliorate localized scleroderma. More recently others have described the topical application of 8% Pirfenidone lotion to dermal scar sites to reduce hypertrophic scarring.

Silicone polymers are well-known to be functional in the topical or transdermal delivery of various APIs.

### SUMMARY OF THE INVENTION

In an effort to improve the art, I have created both wound dressings and scar management products containing Pirfenidone to be delivered to the open wound or transdermally to the closed wound site to reduce and prevent dermal scarring.

### BRIEF DESCRIPTION OF THE DRAWING

FIG. 1 shows schematic representation of a partial view in cross-section of a preferred wound dressing or a preferred scar management dressing constructed in accordance with the invention, in which reference number 15 refers to the layer of the dressing that comprises the wound or skin contacting side or surface of the dressing and reference 13 refers in general to the remainder of the dressing that is shown in FIG. 1.

### DETAILED DESCRIPTION

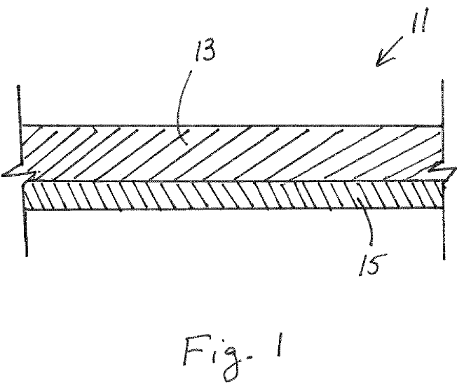

In a preferred embodiment, the invention, as illustrated FIG. 1, comprises a dressing 11, which may be a wound dressing for use on open wounds or a scar management dressing for use on closed wound sites, for reducing and/or preventing dermal scarring. In the preferred embodiment of the invention shown in FIG. 1, the dressing 11 has a layer 15 that comprises the wound or skin contacting side or surface of the dressing 11, and an API (preferably, Pirfenidone) is incorporated in the layer 15 to be delivered to the wound or scar area to reduce and/or prevent dermal scarring.

The following examples are not intended to be limiting, as variations on these designs, configurations and processes would be obvious to those skilled in the art. Likewise, it is believed that other materials and APIs could be used to achieve the same concept of this invention. The following examples illustrate various preferred embodiments of the invention and preferred methods of manufacturing these preferred embodiments of the invention. In these examples, 3% by weight of Pirfenidone was mixed into the silicone formulation used to make the layer 15 of the dressing 11 comprising the wound/skin contacting side or surface of the dressing 11. However, a preferred range of 0.5% to 20% by weight of Pirfenidone may be used for the purpose of this invention by mixing 0.5% to 20% by weight of Pirfenidone into the silicone formulation used to make the layer 15 of the dressing 11, that is, the layer of the dressing comprising the wound/skin contacting side or surface of the dressing. Additionally, this invention could apply to numerous wound dressing and scar management methodologies.

### Example 1

Three percent by weight of Pirfenidone was mixed into the silicone formulation of the wound contacting side of Silon-TSR wound dressing, which is manufactured as described in Dillon U.S. Pat. No. 4,832,009, which is incorporated herein by reference.

### Example 2

Three percent by weight of Pirfenidone was mixed into the silicone formulation of the wound contacting side of Rylon-1 wound dressing.

Specifically, a carrier substrate is passed through a reservoir of uncured liquid silicone having 3% by weight Pirfenidone mixed therewith, and an adjustable blade “knife” is set to meter off excess liquid silicone mixed with Pirfenidone that is on the carrier substrate, thereby leaving a layer of liquid silicone, having 3% by weight Pirfenidone mixed therein, on the carrier substrate. An apertured mesh then is laid down onto and into contact with the layer of liquid silicone having 3% by weight Pirfenidone mixed therein, and the resultant material then is passed through a tunnel style oven to apply heat and effectuate crosslinking of the silicone to form a web of at least partially impregnated apertured mesh material on the carrier substrate. The web then is passed through a die cutting apparatus to punch-cut the final dressing shapes.

### Example 3

Three percent by weight of Pirfenidone was mixed into the silicone formulation of the skin contacting side of Oleeva Fabric scar management dressing, which is manufactured using the technology describe in Dillon U.S. Pat. No. 7,087,135, which is incorporated herein by reference.

### Example 4

Three percent by weight of Pirfenidone is mixed into the silicone formulation of the skin contacting surface of a thermoplastic splinting material, which is manufactured as described in Dillon U.S. Pat. No. 5,759,560, which is incorporated herein by reference.

### Example 5

Three percent by weight of Pirfenidone is mixed into the silicone formulation of the skin contacting side of Silon-TEX compression garment textile material, which is manufactured as described in Dillon U.S. Pat. No. 8,084,051, which is incorporated herein by reference.

In use, the inventive dressing is positioned in place by applying the wound/skin contacting surface of the inventive dressing over and into contact with an open wound or a closed wound site, and is maintained in place over and in contact with the open wound or the closed wound site for an effective amount of time, thereby reducing or preventing dermal scarring. When the inventive dressing is used in conjunction with an open wound, the dressing may be changed and replaced as needed or required with fresh dressings of the invention, to continue delivery of the treatment to the open wound to reduce or prevent dermal scarring. Likewise, when the inventive dressing is used in conjunction with a closed wound site, the dressing may be changed and replaced as needed or required with fresh dressings of the invention, to continue delivery of the treatment to the closed wound site to reduce or prevent dermal scarring.

1. A scar management product for reducing and/or preventing dermal scarring, comprising
a skin contacting layer having a skin contacting side, and
pirfenidone incorporated into the skin contacting layer.

2. The scar management product of claim, 1
the skin contacting layer comprising 0.5% to 20% by weight pirfenidone.

3. The scar management product of claim 1,
the skin contacting layer comprising 3% by weight pirfenidone.

4. The scar management product of claim 1,
the skin contacting layer comprising a silicone formulation.

5. The scar management product of claim 4,
the silicone formulation comprising 0.5% to 20% by weight pirfenidone.

6. The scar management product of claim 4,
the silicone formulation comprising 3% by weight pirfenidone.

7. The scar management product of claim 4,
the silicone formulation being silicone.

8. The scar management product of claim 4,
the silicone formulation being polyorganosiloxane.

9. The scar management product of claim 4,
the silicone formulation being polydimethylsiloxane.

10. The scar management product of claim 1, further including
a remainder portion of the scar management product positioned on the skin contacting layer,
the remainder portion including textile fabric, foam, non-woven film, mesh, thermoplastic material, or a material other than a textile fabric.

11. A method of manufacturing a scar management product, comprising the steps of
mixing pirfenidone into a silicone formulation to form a mixture thereof,
the silicone formulation comprising polyorganosiloxane, silicone, silicone gel, silicone elastomer, or polydimethylsiloxane,
applying the mixture onto a carrier to form a layer of the mixture thereon, and
curing the layer of the mixture.

12. The method of claim 11, further including
applying expanded polytetrafluoroethylene to the layer of the mixture prior to curing.

13. The method of claim 12,
the silicone formulation being silicone,
the layer of the mixture and the expanded polytetrafluoroethylene forming an interpenetrating polymer network of silicone and polytetrafluoroethylene, the interpenetrating polymer network having a first side and a second side, the first side forming the skin contacting side of the skin contacting layer, and further including the step of
laminating thermoplastic splinting material to the second side of the interpenetrating polymer network.

14. The method of claim 11, further including
applying an apertured mesh layer to the layer of the mixture prior to curing.

15. The method of claim 11,
further including, prior to curing, the steps of
applying a microporous polymer sheeting membrane to the layer of the mixture and allowing or causing the layer of the mixture to impregnate the microporous polymer sheeting membrane, and
applying a backing material to the distal surface of the impregnated microporous polymer sheeting material, with the backing material being in contact with the mixture that has made its way through the microporous polymer sheeting membrane.

16. The method of claim 15,
the backing material comprising textile fabric, foam, non-woven film, or material other than a textile fabric.

17. The method of claim 11, further including, prior to curing, the steps of
applying a lamination of a microporous polymer sheeting membrane and backing material to the layer of the mixture and allowing or causing the layer of the mixture to impregnate the microporous polymer sheeting membrane.

18. The method of claim 17,
the backing material comprising textile fabric, foam, non-woven film, or material other than a textile fabric.

19. A method of manufacturing a scar management product, comprising the steps of
mixing pirfenidone into a silicone formulation to form a mixture thereof,
applying the mixture onto a textile fabric to form a textile fabric having the mixture applied thereto, and
fabricating the textile fabric having the mixture applied thereto into a scar management product.

20. The method of claim 19,
the scar management producing being a garment.

21. A method of reducing dermal scars, comprising the steps of
providing the scar management product of claim 1,
applying the skin contacting side of the skin contacting layer of the scar management product over and into contact with a closed wound site, and
maintaining the scar management product over and into contact with the closed wound site for an effective amount of time to reduce and/or prevent dermal scarring.
